SEQUENCE LISTING (40 sequences)

SEQ ID NO: 1 - protein, 12 aa, Artificial Sequence
  MDM2 sequence of the interaction domain between MDM4 and MDM2 from aa 427 to aa 438 of MDM2
ESSLPLNAIEPC

SEQ ID NO: 2 - protein, 12 aa, Artificial Sequence
  MDM4 sequence of the interaction domain between MDM4 and MDM2 from aa 427 to aa 438 of MDM4
MEDCQNLLKPCS

SEQ ID NO: 3 - protein, 12 aa, Artificial Sequence
  MDM4 sequence of the interaction domain between MDM4 and MDM2 from aa 479 to aa 490 of MDM4
KEIQLVIKVFIA

SEQ ID NO: 4 - protein, 490 aa, Artificial Sequence
  Human MDM4 protein sequence
MTSFSTSAQCSTSDSACRISPGQINQVRPKLPLLKILHAAGAQGEMFTVKEVMHYLGQYIMVKQLYDQQEQHMVYCGGDLLGELLGRQSFSVKDPSPLYDMLRKNLVTLATATTDAAQTLALAQDHSMDIPSQDQLKQSAEESSTSRKRTTEDDIPTLPTSEHKCIHSREDEDLIENLAQDETSRLDLGFEEWDVAGLPWWFLGNLRSNYTPRSNGSTDLQTNQDVGTAIVSDTTDDLWFLNESVSEQLGVGIKVEAADTEQTSEEVGKVSDKKVIEVGKNDDLEDSKSLSDDTDVEVTSEDEWQCTECKKFNSPSKRYCFRCWALRKDWYSDCSKLTHSLSTSDITAIPEKENEGNDVPDCRRTISAPVVRPKDAYIKKENSKLFDPCNSVEFLDLAHSSESQETISSMGEQLDNLSEQRTDTENMEDCQNLLKPCSLCEKRPRDGNIIHGRTGHLVTCFHCARRLKKAGASCPICKKEIQLVIKVFIA

SEQ ID NO: 5 - protein, 78 aa, Artificial Sequence
  MDM4 sequence of the interaction domain between MDM4 and MDM2 from aa 413 to aa 490 of MDM4
QLDNLSEQRTDTENMEDCQNLLKPCSLCEKRPRDGNIIHGRTGHLVTCFHCARRLKKAGASCPICKKEIQLVIKVFIA

SEQ ID NO: 6 - protein, 63 aa, Artificial Sequence
  MDM4 sequence of the interaction domain between MDM4 and MDM2 from aa 428 to aa 490 of MDM4
EDCQNLLKPCSLCEKRPRDGNIIHGRTGHLVTCFHCARRLKKAGASCPICKKEIQLVIKVFIA

SEQ ID NO: 7 - protein, 491 aa, Artificial Sequence
  Human MDM2 protein sequence
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 8 - protein, 81 aa, Artificial Sequence
  MDM2 sequence of the interaction domain between MDM4 and MDM2 from aa 411 to aa 491 of MDM2
VKEFEREETQDKEESVESSLPLNAIEPCVICQGRPKNGCIVHGKTGHLMACFTCAKKLKKRNKPCPVCRQPIQMIVLTYFP

SEQ ID NO: 9 - protein, 64 aa, Artificial Sequence
  MDM2 sequence of the interaction domain between MDM4 and MDM2 from aa 428 to aa 491 of MDM2
SSLPLNAIEPCVICQGRPKNGCIVHGKTGHLMACFTCAKKLKKRNKPCPVCRQPIQMIVLTYFP

SEQ ID NO: 10 - protein, 18 aa, Artificial Sequence
  MDM4 sequence of the interaction domain between MDM4 and MDM2 from aa 473 to aa 490 of MDM4
SCPICKKEIQLVIKVFIA

SEQ ID NO: 11 - protein, 31 aa, Artificial Sequence
  MDM4 sequence of the interaction domain between MDM4 and MDM2 from aa 418 to aa 448 of MDM4
SEQRTDTENMEDCQNLLKPCSLCEKRPRDGN

SEQ ID NO: 12 - protein, 35 aa, Artificial Sequence
  MDM2 sequence of the interaction domain between MDM4 and MDM2 from aa 415 to aa 449 of MDM2
EREETQDKEESVESSLPLNAIEPCVICQGRPKNGC

SEQ ID NO: 13 - protein, 4 aa, Artificial Sequence
  MDM4 sequence of the interaction domain between MDM4 and MDM2 from aa 82 to aa 85 of MDM4
QLVI

SEQ ID NO: 14 - protein, 5 aa, Artificial Sequence
  MDM4 sequence of the interaction domain between MDM4 and MDM2 from aa 81 to aa 85 of MDM4
IQLVI

SEQ ID NO: 15 - protein, 6 aa, Artificial Sequence
  MDM4 sequence of the interaction domain between MDM4 and MDM2 from aa 80 to aa 85 of MDM4
EIQLVI

SEQ ID NO: 16 - protein, 7 aa, Artificial Sequence
  MDM4 sequence of the interaction domain between MDM4 and MDM2 from aa 79 to aa 85 of MDM4
KEIQLVI

SEQ ID NO: 17 - protein, 8 aa, Artificial Sequence
  MDM4 sequence of the interaction domain between MDM4 and MDM2 from aa 78 to aa 85 of MDM4
KKEIQLVI

SEQ ID NO: 18 - protein, 9 aa, Artificial Sequence
  MDM4 sequence of the interaction domain between MDM4 and MDM2 from aa 77 to aa 85 of MDM4
CKKEIQLVI

SEQ ID NO: 19 - protein, 10 aa, Artificial Sequence
  MDM4 sequence of the interaction domain between MDM4 and MDM2 from aa 76 to aa 85 of MDM4
ICKKEIQLVI

SEQ ID NO: 20 - protein, 11 aa, Artificial Sequence
  MDM4 sequence of the interaction domain between MDM4 and MDM2 from aa 75 to aa 85 of MDM4
PICKKEIQLVI

SEQ ID NO: 21 - protein, 12 aa, Artificial Sequence
  MDM4 sequence of the interaction domain between MDM4 and MDM2 from aa 74 to aa 85 of MDM4
CPICKKEIQLVI

SEQ ID NO: 22 - protein, 4 aa, Artificial Sequence
  MDM2 sequence of the interaction domain between MDM4 and MDM2 from aa 26 to aa 29 of MDM2
VESS

SEQ ID NO: 23 - protein, 5 aa, Artificial Sequence
  MDM2 sequence of the interaction domain between MDM4 and MDM2 from aa 25 to aa 29 of MDM2
SVESS

SEQ ID NO: 24 - protein, 6 aa, Artificial Sequence
  MDM2 sequence of the interaction domain between MDM4 and MDM2 from aa 24 to aa 29 of MDM2
ESVESS

SEQ ID NO: 25 - protein, 7 aa, Artificial Sequence
  MDM2 sequence of the interaction domain between MDM4 and MDM2 from aa 23 to aa 29 of MDM2
EESVESS

SEQ ID NO: 26 - protein, 8 aa, Artificial Sequence
  MDM2 sequence of the interaction domain between MDM4 and MDM2 from aa 22 to aa 29 of MDM2
KEESVESS

SEQ ID NO: 27 - protein, 9 aa, Artificial Sequence
  MDM2 sequence of the interaction domain between MDM4 and MDM2 from aa 21 to aa 29 of MDM2
DKEESVESS

SEQ ID NO: 28 - protein, 10 aa, Artificial Sequence
  MDM2 sequence of the interaction domain between MDM4 and MDM2 from aa 20 to aa 29 of MDM2
QDKEESVESS

SEQ ID NO: 29 - protein, 11 aa, Artificial Sequence
  MDM2 sequence of the interaction domain between MDM4 and MDM2 from aa 19 to aa 29 of MDM2
TQDKEESVESS

SEQ ID NO: 30 - protein, 4 aa, Artificial Sequence
  MDM2 sequence of the interaction domain between MDM4 and MDM2 from aa 35 to aa 38 of MDM2
IEPC

SEQ ID NO: 31 - protein, 5 aa, Artificial Sequence
  MDM2 sequence of the interaction domain between MDM4 and MDM2 from aa 35 to aa 39 of MDM2
IEPCV

SEQ ID NO: 32 - protein, 6 aa, Artificial Sequence
  MDM2 sequence of the interaction domain between MDM4 and MDM2 from aa 35 to aa 40 of MDM2
IEPCVI

SEQ ID NO: 33 - protein, 7 aa, Artificial Sequence
  MDM2 sequence of the interaction domain between MDM4 and MDM2 from aa 35 to aa 41 of MDM2
IEPCVIC

SEQ ID NO: 34 - protein, 8 aa, Artificial Sequence
  MDM2 sequence of the interaction domain between MDM4 and MDM2 from aa 35 to aa 42 of MDM2
IEPCVICQ

SEQ ID NO: 35 - protein, 9 aa, Artificial Sequence
  MDM2 sequence of the interaction domain between MDM4 and MDM2 from aa 35 to aa 43 of MDM2
IEPCVICQG

SEQ ID NO: 36 - protein, 10 aa, Artificial Sequence
  MDM2 sequence of the interaction domain between MDM4 and MDM2 from aa 35 to aa 44 of MDM2
IEPCVICQGR

SEQ ID NO: 37 - protein, 11 aa, Artificial Sequence
  MDM2 sequence of the interaction domain between MDM4 and MDM2 from aa 35 to aa 45 of MDM2
IEPCVICQGRP

SEQ ID NO: 38 - protein, 12 aa, Artificial Sequence
  MDM4 sequence of the interaction domain between MDM4 and MDM2 from aa 479 to aa 490 of MDM4 in which FI in positions 10 and 11 of said sequence has been substituted with AE
KEIQLVIKVAEA

SEQ ID NO: 39 - protein, 12 aa, Artificial Sequence
  peptide composed with the same aminoacids of SEQ ID NO3 but arranged in different order
VQEAFKLIKIVI

SEQ ID NO: 40 - protein, 12 aa, Artificial Sequence
  peptide composed with the same aminoacids of SEQ ID NO3 but arranged in different order
AIKIFVKVLEIQ

(19) United States
(12) Patent Application Publication
(10) Pub. No.: US 2013/0217634 A1
(43) Pub. Date: Aug. 22, 2013

(54) PEPTIDES ABLE TO INTERFERE WITH THE INHIBITING ACTIVITY OF MDM2/MDM4 HETERODIMER TOWARDS P53 AND USE THEREOF FOR CANCER TREATMENT

(71) Applicants: CONSIGLIO NAZIONALE DELLE RICERCHE, (US); UNIVERSITA' DEGLI STUDI DI PERUGIA, (US)

(72) Inventors: FABIOLA MORETTI, ROMA (RM) (IT); FRANCESCA MANCINI, ROMA (RM) (IT); MARSHA PELLEGRINO, ROMA (RM) (IT); ANTONIO MACCHIARULO, PERUGIA (PG) (IT); ROBERTO PELLICCIARI, PERUGIA (PG) (IT)

(73) Assignees: UNIVERSITA' DEGLI STUDI DI PERUGIA, PERUGIA (PG) (IT); CONSIGLIO NAZIONALE DELLE RICERCHE, ROMA (RM) (IT)

(21) Appl. No.: 13/773,377

(22) Filed: Feb. 21, 2013

(30) Foreign Application Priority Data
Feb. 21, 2012 (IT) RM2012A000060

(51) Int. Cl. C07K 7/08

(52) U.S. Cl. CPC C07K 7/08
USPC 514/19.2; 530/327; 536/23.5; 435/320.1; 514/21.5; 514/44 R; 436/501

(57) ABSTRACT

The present disclosure concerns peptides able to interfere and in particular impair the inhibiting activity of MDM2/MDM4 heterodimer towards p53 and maintain the association between MDM4 and p53 so to restore the p53 oncosuppressive function in cancer cells harboring wild type p53 protein, directing its function specifically towards an apoptotic outcome.

### CROSS REFERENCE TO RELATED APPLICATIONS

The present application claims priority to Italian Patent Application No. RM2012000060, file on Feb. 21, 2012, the content of which is hereby incorporated by reference in its entirety.

### FIELD

The present disclosure relates to p53 protein and in particular to compounds able to interfere with the activity of the MDM2/MDM4 heterodimer

### BACKGROUND

The MDM2 and MDM4 are oncoproteins which are key negative regulators of the p53 tumor suppressor. Despite, several studies on the MDM2 and MDM4 contributions to the regulation of p53 stability and activity, development of approaches able to interfere with the activity of the MDM2 and MDM4 oncoproteins is still challenging.

### SUMMARY

Provided herein are peptides able to interfere with the MDM2/MDM4 heterodimer activity on p53.

In particular, a peptide having sequence of MDM2 or MDM4 interaction domain is described, as well as related nucleotide sequences, expression vectors, pharmaceutical compositions, medicaments, methods and systems.

### BRIEF DESCRIPTION OF THE DRAWINGS

The present disclosure now will be described by an illustrative, but not limitative way, according to preferred embodiments thereof, with detailed reference to enclosed drawings, wherein:

FIG. 1 shows cell death analysis as a result of over-expression of MDM4+p53, or MDM4C437G+p53 in the presence or absence of MDM2, in embryonic murine Mdm4^{-/-} p53^{-/-} (DNX) fibroblasts compared to control cells transfected using an empty vector (null). The percentage of dead cells has been calculated as ratio of Trypan blue positive cells to all counted cells. The average of 2 different experiments is reported.

FIG. 2 shows the binding interface of the two MDM4 and MDM2 RING finger domains. Boxed (in MDM4) and circled (in MDM2) positions represent potential key amino acids for this interaction.

FIG. 3 shows the binding analysis of MDM2 to various MDM4 point mutants. DNX cells were transfected with expression vectors carrying MDM2-wt and MDM4-wt or its mutated forms (MDM4 L433N, L433D, I489Q, I489E) as shown in the upper part of the figure. Cell lysates have been collected and 700 μg of whole cell extracts have been immunoprecipitated with anti-MDM2 antibody and immunocomplexes analyzed by western blotting, as indicated in the figure (right panel). Left panel shows western blot analysis of a fraction of cellular lysates in order to control the expression levels of reported proteins.

FIG. 4 shows the binding analysis of MDM4 to various MDM2point mutants. Murine embryonic Mdm2^{-/-}p53^{-/-} (DN1) fibroblasts were transfected with expression vectors carrying MDM4-wt and MDM2-wt or its mutated forms (MDM2-L430Q, L430E, A434N, A434D) as shown in the upper part of the figure. Cell lysates have been collected and 700 μg of whole cell extracts have been immunoprecipitated with anti-MDM4 antibody and immunocomplexes analyzed by western blotting, as indicated in the figure (right panel). Left panel shows western blot analysis of a fraction of cellular lysates in order to control the expression levels of reported proteins.

FIG. 5 shows the ability of peptide 1 to dissociate MDM4/MDM2 complex by immunoprecipitation assay. Transient transfection of peptides has been carried out in HCT116 wt and p53−/− colon carcinoma cell lines and the immunoprecipitation of MDM4 has been carried out on cell lysates in order to detect the presence of MDM2 within the immunocomplex, by western blot analysis.

FIG. 6 shows the ability of peptide 1 to induce cell death. Cytofluorimetric analysis was carried out on HCT116 wt and p53−/− cells transfected with FITC tagged peptides and stained with Propidium Iodide (PI). Percentage of dead cells was evaluated specifically among the transfected cells (green population).

FIG. 7 shows the ability of peptide 3 to dissociate MDM4/MDM2 complex by immunoprecipitation assay. Transient transfection of peptides has been carried out in HCT116 wt and p53−/− colon carcinoma cell lines and the immunoprecipitation of MDM4 has been performed on cell lysates in order to detect the presence of MDM2 within the immunocomplex by western blot analysis.

FIG. 8 shows the ability of peptide 3 to induce cell death. Cytofluorimetric analysis was performed on HCT116 wt and p53−/− cells transfected with FITC tagged peptides and stained with Propidium Iodide (PI). Percentage of dead cells was evaluated specifically among the transfected cells (green population).

FIG. 9 shows in vitro ubiquitination assay of p53 performed by recombinant GST-MDM2. The figure shows the ability of Peptide 3, compared to control peptides (3D, SC3A and SC3B), to inhibit the ubiquitinatin activity of MDM2 towards p53.

FIG. 10 shows the ability of peptide 3, compared to control peptides (3D and 2MUT), to induce apoptosis. In order to specifically detect apoptotic cells, TUNEL assay was carried out in HCT116 wt and p53−/− cells transfected with FITC tagged peptides. Percentage of apoptotic cells was evaluated specifically among the transfected cells (green population).

FIG. 11 shows the binding mode of peptide 3 to MDM2 as resulting from molecular dynamic simulations. I489 occupies a hydrophobic cleft composed of A434, I435 and L458 of MDM2. F488 forms aromatic interactions with a H457 of MDM2 protein.

### DETAILED DESCRIPTION OF PREFERRED EMBODIMENTS

The present disclosure concerns peptides able to interfere and in particular impair the inhibiting activity of MDM2/MDM4 heterodimer towards p53 and their use for cancer treatment. In particular, the disclosure concerns peptides able to inhibit MDM2/MDM4 heterodimer and maintain the association between MDM4 and p53 so to restore the p53 oncosuppressive function in cancer cells harboring wild type p53 protein, directing its function specifically towards an apoptotic outcome.

Inactivation of the oncosuppressor p53 by means of direct mutation of its gene is one of first genetic lesions recognized in human tumors (about 50% of human tumors). In tumors carrying wilde type p53, studies have evidenced alternative methods for p53 inactivation by deregulation of its partners, MDM4 and MDM2. The latter ones are two crucial regulators of the oncosuppressor p53. These two proteins carry out mainly inhibiting activity towards p53 by controlling both protein levels and transcriptional function.

The importance of MDM proteins on the inhibition of p53 activity resulted in development of different strategies aiming to disrupt the p53/MDM association.

For example WO 2008106507, WO201105219 patent applications are known and concern peptides inhibiting the interaction of p53 with MDM2 and MDM4, i.e. peptides aiming to break p53 binding with MDM4 and MDM2. According to WO201105219 patent application, the dissociation of p53 from MDM2 and MDM4 results in a biological response consisting both of proliferative arrest and cell death, although it is not specified whether the observed cell death is due to apoptosis.

WO2008106507 describes peptides able to bind with high affinity MDM2 and therefore, probably, inhibit the binding of MDM2 with all the members of the p53 family; however, such statement has not been experimentally proved. It is predicted, by computational analysis, that such peptides may bind MDM4 and therefore dissociate the same from p53. In vitro or in vivo experimental data have not been provided for a possible biological activity of these peptides. Therefore it is not predictable their effectiveness.

In addition, the strategy aiming to disrupt the binding of p53 to MDM4 and MDM2 does not consider recent studies suggesting that MDM4 binding to p53 potentiates its apoptotic activity. According to WO201105219 patent application, peptides would be able to inhibit the binding of p53 to MDM2 10 fold higher than to MDM4 (IC50=0.01 μM and 0.1 μM for MDM2 and MDM4, respectively). Therefore, it cannot be excluded that a part of the observed cell death, according to WO201105219, is due to the partial retention of MDM4-p53 complexes and not completely to the dissociation of p53 from MDM2 and MDM4.

WO200061193 patent application describes antisense compounds inhibiting MDM4 expression. Also in this case the importance of MDM4-p53 complex has not been taken into consideration for apoptosis induction.

Most promising results for reactivation of p53 are obtained using small molecules identified by high-throughput screening of compound libraries and drug design by computer analysis based on protein structure (Shangary S, Wang S. Targeting the MDM2-p53 interaction for cancer therapy. Clin Cancer Res. 2008 Sep. 1; 14(17): 5318-24).

However, known therapeutic strategies show various disadvantages. For example, compounds dissociating MDM2 from p53 are ineffective in displacing MDM4 from p53 because of the intrinsic differences in p53 binding domains of MDM4 and MDM2. This could enhance apoptosis induction by MDM4-p53 complex. However, since such compounds do not inhibit the binding of MDM4 with MDM2, it can be predicted that the heterodimer still inhibits p53 activity. Moreover, association of MDM2 to MDM4 antagonizes the pro-apoptotic activity of MDM4 preventing this response. In addition growth arrest rather than apoptotic response often occurs, while thepreferred result in any cancer therapy is apoptosis (Toledo F, Wahl G M. MDM2 and MDM4: p53 regulators as targets in anticancer therapy. Int J Biochem Cell Biol. 2007; 39 (7-8): 1476-82. Epub 2007 Apr. 8; Shangary S, Wang S. Targeting the MDM2-p53 interaction for cancer therapy. Clin Cancer Res. 2008 Sep. 1; 14(17): 5318-24). Known methods, aiming to inhibit the interaction of p53 with MDM2 and/or MDM4, cause therefore activation of p53 not only towards apoptosis, as it would be preferred, but also towards proliferative arrest. In addition, the release of MDM2 from association with p53 results in higher degradative activity of MDM2 towards MDM4 and other proapoptotic molecules (as demonstrated by several recent papers).

All current methods failed to pass phase 1 of clinical trials, pointing out to existing limitations (Cheok C F, Verma C S, Baselga J, Lane D P. Translating p53 into the clinic. Nat Rev Clin Oncol. 2011 January; 8(1):25-37. Epub 2010 Oct. 26).

Based on these results, it emerges the need to provide new therapeutic methods for cancer treatment that overcomes the disadvantages of known methods.

Recent studies suggest that the MDM4/MDM2 heterodimer is the functional inhibitor of p53. This data suggest that agents aiming to inhibit MDM4/MDM2 heterodimer could represent new potential therapeutic agents. On the other hand, recent data suggest different activities of MDM2 and MDM4 under stress conditions. Under these conditions a weakening of association and degradation activity of MDM2 towards p53 mainly occurs (Marchenko ET to, 2007). In addition, upon a sub-lethal type of DNA damage, usually associated with growth arrest, MDM2 level increase and this results in degradation of MDM4 and some pro-apoptotic factors as HIPK2 (Rinaldo C, Prodosmo A, Mancini F, Iacovelli S, Sacchi A, Moretti F, Soddu S. MDM2-regulated degradation of HIPK2 prevents p53Ser46 phosphorylation and DNA damage-induced apoptosis. Mol Cell. 2007 Mar. 9; 25(5):739-50). On the contrary, following a severe DNA damage, usually associated to apoptosis, levels of MDM2 do not increase or even decrease (Ashcroft M, Taya Y, Vousden K H Stress signals utilize multiple pathways to stabilize p53. Mol Cell Biol 200020: 3224-33; Latonen L, Taya Y, Laiho M UV-radiation induces dose-dependent regulation of p53 response and modulates p53-HDM2 interaction in human fibroblasts. Oncogene 2001 20: 6784-93; Meng L H, Kohn K W, Pommier Y Dose-response transition from cell cycle arrest to apoptosis with selective degradation of Mdm2 and p21WAF1/CIP1 in response to the novel anticancer agent, aminoflavone (NSC 686,288). Oncogene. 2007, 26, 4806-16).

Based on these data, a recent model considers the up-regulation of MDM2 occurring during cell growth arrest, an event favoring this response in comparison to cell death (Shmueli A, Oren M. Mdm2: p53's lifesaver? Mol Cell. 2007 Mar. 23; 25(6):794-6). On the other hand, MDM4 stabilizes p53, preventing the MDM2-mediated degradation, and promotes p53 mediated apoptosis under stress conditions (Jackson M W, Berberich S J. MdmX protects p53 from Mdm2-mediated degradation. Mol Cell Biol 2000; 20:1001-7; Stad R, Little N A, Xirodimas D P, Frenk R, van der Eb A J, Lane D P, et al. Mdmx stabilizes p53 and Mdm2 via two distinct mechanisms. EMBO Rep 2001; 2:1029-34; Mancini F, Gentiletti F, D'Angelo M, Giglio S, Nanni S, D'Angelo C, et al. MDM4 (MDMX) over-expression enhances stabilization of stress-induced p53 and promotes apoptosis. J Biol Chem 2004, 279:8169-80; Barboza J A, Iwakuma T, Terzian T, El-Naggar A K, Lozano G. Mdm2 and Mdm4 loss regulates distinct p53 activities. Mol Cancer Res 2008; 6:947-54).

Therefore, although it is known that MDM2/MDM4 heterodimer inhibits p53 function, heterodimer dissociation has not been assumed a current suitable strategy in order to favor p53 apoptotic activity. This due to the fact that MDM2 release, i.e. the main p53 inhibitor, could however carry out inhibiting function against p53. For this reason, current strategies are directed to the dissociation of p53 from MDM2 and MDM4 and not to hetorodimer dissociation.

Applicants have demonstrated that MDM2 opposes the pro-apoptotic activity of MDM4 and that, when MDM4/MDM2 dissociation or partial dissociation at specific binding sites occurs as result of stress conditions, the p53-mediated apoptotic pathway is promoted, independently of MDM2. These results are coherent with the model proposed by Shmueli indicating p53 regulators not only for control of p53 activity, but also as having an important role in p53-mediated choice between cell life and death (Shmueli A, Oren M. Mdm2: p53's lifesaver? Mol Cell. 2007 Mar. 23; 25(6):794-6; Mitochondrial MDM4 (MDMX): An unpredicted role in the p53-mediated intrinsic apoptotic pathway. Mancini F, Moretti F. Cell Cycle. 2009 December; 8(23):3854-9). In particular, the inventors of the present disclosure observed that the over-expression of MDM4 mutant (C437G), unable of binding MDM2, induces the activation of p53, and this activation of p53 is not inhibited by concomitant expression of MDM2. Moreover, the over-expression of MDM4 (exceeding MDM2 levels) in a model of transgenic mouse causes an increase of cellular death (apoptosis) as a result of gamma irradiation and delays the tumor appearance induced by mouse exposure to genotoxic agents.

On the basis of these data, MDM4/MDM2 interaction region has been characterized. Amino acids involved in MDM4/MDM2 binding have been identified and tested by molecular mutagenesis. L430, A434 in MDM2 and L433, I489, F488 residues in MDM4, respectively, have been identified as relevant for MDM2/MDM4 complex.

Therefore, peptides belonging to the region of MDM4/MDM2 interaction and able to impair the inhibiting activity of MDM2/MDM4 heterodimer towards p53, that is to restore the p53 oncosuppressive function in cancer cells still harboring p53 wild type protein (approximately 50% of human tumors), and to maintain the proapoptotic function of MDM4 directing therefore the p53 response towards the apoptosis, have been prepared. Currently the majority of human tumors is characterized in that 50% express wild type and 50% express mutated p53, respectively. Therefore, independently from tumor type, this approach is directed to tumors expressing wild type p53.

In particular, three peptides of 12 amino acids (12 mer) have been prepared and tested at molecular and biological level. Since the peptides display some limitations for therapeutic application because of intrinsic instability thereof, chemical modifications necessary to increase the bioavailability and allow the in vivo administration have been carried out.

Techniques in order to increase the stability and bioavailability of peptides and allow administration thereof in vivo are well known to those skilled in the art. Particularly, above said peptides could be modified by means of insertion of hydrocarbon bridge suitable to increase the alpha helicity of said peptides (carbon staples). These modifications are able to promote cellular uptake, confer resistance to protease and increase global stability.

It is therefore a specific object of the present disclosure a peptide belonging to (i.e. comprised in, taking part of) MDM4 or MDM2 sequence, at the levels of the interaction domain between MDM4 and MDM2, or mimetic peptides of said peptide, said peptide having a number of amino acids from 5 to 16, preferably from 8 to 12, and comprising at least one amino acid chosen in the group consisting of leucine, isoleucine or phenylalanine of MDM4 position 433, 489 or 488, respectively, or comprising at least one amino acid chosen in the group consisting of leucine or alanine of MDM2 position 430 or 434, respectively,

wherein said MDM4 sequence, at the levels of MDM4 and MDM2 interaction domain, is chosen between the following sequences from amino acid 473 to amino acid 490 and from amino acid 418 to amino acid 448, respectively:

| (SEQ ID NO: 10) | |
|---|---|
| 473 | SCPICKKE IQLVIKVFIA 490 |
| (SEQ ID NO: 11) | |
| 418 | SEQ RTDTENMEDC QNLLKPCSLC EKRPRDGN 448, |
| SEQ ID NO: 10 | |
| | preferably; |

and said MDM2 sequence, at the levels of MDM4 and MDM2 interaction domain, is the following sequence from amino acid 415 to amino acid 449:

(SEQ ID NO: 12)

| | |
|---|---|
| 415 | EREETQ DKEESVESSL PLNAIEPCVI CQGRPKNGC 449. |

Preferably, when said peptide belongs to MDM4, the peptide according to the disclosure comprises the aminoacids isoleucine and phenylalanine of MDM4 position 489 and 488, respectively.

According to a preferred embodiment, the present disclosure concerns a peptide or mimetic peptides thereof according to the disclosure, wherein said peptide has the following general formula I:

R-KVFI-R1 (I)

wherein

R is chosen from the group consisting of I, VI, LVI, QLVI (SEQ ID NO:13), IQLVI (SEQ ID NO:14), EIQLVI (SEQ ID NO:15), KEIQLVI (SEQ ID NO:16), KKEIQLVI (SEQ ID NO:17), CKKEIQLVI (SEQ ID NO:18), ICKKEIQLVI (SEQ ID NO:19), PICKKEIQLVI (SEQ ID NO:20), CPICKKEIQLVI (SEQ ID NO:21) or hydrogen of amino functional group of K;

R1 is chosen from the group consisting of A or hydroxyl of carboxylic functional group of I;

wherein said R and R1 are chosen so that said peptide ranges from 5 to 16 amino acids, preferably, from 8 to 12. Preferably, the peptide belonging to the sequence of MDM4, at the levels of MDM4 and MDM2 interaction domain, can consist of the following sequence from amino acid 479 to amino acid 490 of MDM4 or mimetic peptides thereof:

| | |
|---|---|
| KEIQLVIKVFIA. | (SEQ ID NO: 3) |

Again according to a preferred embodiment, the present disclosure concerns a peptide or mimetic peptides thereof according to the disclosure, wherein said peptide has the following general formula II:

R2-LPLNA-R3 (II)

wherein

R2 is chosen from the group consisting of S, SS, ESS, VESS (SEQ ID NO:22), SVESS (SEQ ID NO:23), ESVESS (SEQ ID NO:24), EESVESS (SEQ ID NO:25), KEESVESS (SEQ ID NO:26), DKEESVESS (SEQ ID NO:27), QDKEESVESS (SEQ ID NO:28), TQDKEESVESS (SEQ ID NO:29) or hydrogen of amino functional group of L;

R3 is chosen from the group consisting of I, IE, IEP, IEPC (SEQ ID NO:30), IEPCV (SEQ ID NO:31), IEPCVI (SEQ ID NO:32), IEPCVIC (SEQ ID NO:33), IEPCVICQ (SEQ ID NO:34), IEPCVICQG (SEQ ID NO:35), IEPCVICQGR (SEQ ID NO:36), IEPCVICQGRP (SEQ ID NO:37) or hydroxyl of carboxylic functional group of A;

wherein said R2 and R3 are chosen so that said peptide ranges from 5 to 16 amino acids, preferably, from 8 to 12. Preferably, the peptide belonging to the sequence of MDM2, at the levels of MDM4 and MDM2 interaction domain, can consist of the following sequence from amino acid 427 to amino acid 438 of MDM2 or mimetic peptides thereof:

| | |
|---|---|
| ESSLPLNAIEPC | (SEQ ID NO: 1) |

An isolated nucleotide sequence encoding for peptide as above defined is a further object of the present disclosure.

Moreover, the disclosure concerns an expression vector comprising nucleotide sequence as above defined.

The disclosure refers also to a pharmaceutical composition comprising or consisting of at least one peptide or mimetic peptides thereof, or at least one nucleotide sequence, or at least one vector as above defined, as an active ingredient, in association with one or more pharmaceutically acceptable adjuvant and/or excipients.

A peptide or mimetic peptides thereof, nucleotide sequence, vector or pharmaceutical composition, as defined above, for use in the treatment of tumors preferably expressing wild type p53, or associated pathologies like hyperproliferative benign syndromes represent a further object of the present disclosure.

Said peptides according to the disclosure can, for example, be administered by intravenous way, or using liposomal vectors directly deliverable to the tumor, or, at last, by means of nano-particles. In addition, said peptides can be delivered also by gene therapy using DNA expression vectors.

As above reported, peptides of the disclosure can be modified according to known methods in order to increase stability and bioavailability of the peptides and allow in vivo administration thereof. Particularly, said peptides can be modified by insertion of hydrocarbon bridge suitable to increase alpha helicity thereof (carbon staples). As reported in experimental section, peptides have been modified as below:

| | |
|---|---|
| peptide 1: | FITC-Ahx-ESSLPLNAIEPS-CONH2 |
| peptide 2: | FITC-Ahx-MEDSQNLLKPSS- CONH2 |
| peptide 3: | FITC-Ahx-KEIQLVIKVFIA-CONH2 |

wherein the Ahx term indicates a linker. In addition, the peptides have been modified, for example, also with acetyl in the place of FITC-Ahx:

| | |
|---|---|
| peptide 1: | Acet - ESSLPLNAIEPS-CONH2 |
| peptide 2: | Acet - MEDSQNLLKPSS- CONH2 |
| peptide 3: | Acet- KEIQLVIKVFIA-CONH2 |

In addition, the present disclosure concerns the use of interaction domain between MDM4 and MDM2 for the screening of compounds able to dissociate at least one amino acid chosen in the group consisting of leucine, isoleucine or phenylalanine of MDM4 position 433, 489 or 488, respectively, or at least one amino acid chosen in the group consisting of leucine or alanine of MDM2 position 430 or 434, respectively. The compounds selected by the above screneening method are antitumoral compounds.

MDM4 sequence of interaction domain between MDM4 and MDM2 can consists of the following MDM4 sequence from amino acid 413 to amino acid 490:

(SEQ ID NO: 5)

| | |
|---|---|
| 413 | QLDNLSEQ RTDTENMEDC QNLLKPCSLC EKRPRDGNII |
| | HGRTGHLVTC FHCARRLKKA GASCPICKKE IQLVIKVFIA |
| | 490 |

or the following MDM4 sequence from amino acid
428 to amino acid 490:
(SEQ ID NO: 6)

| | |
|---|---|
| 428 | EDC QNLLKPCSLC EKRPRDGNII HGRTGHLVTC |
| | FHCARRLKKA GASCPICKKE IQLVIKVFIA 490 |

MDM2 sequence of interaction domain between MDM4 and MDM2 can consists of the following MDM2 sequence from amino acid 411 to amino acid 491:

(SEQ ID NO: 8)

| | |
|---|---|
| 411 | VKEFEREETQ DKEESVESSL PLNAIEPCVI CQGRPKNGCI |
| | VHGKTGHLMA CFTCAKKLKK RNKPCPVCRQ PIQMIVLTYF |
| | P 491 |

or the following MDM2 sequence from amino acid 428 to amino acid 491:

(SEQ ID NO: 9)

| | |
|---|---|
| 428 | SSL PLNAIEPCVI CQGRPKNGCI VHGKTGHLMA |
| | CFTCAKKLKK RNKPCPVCRQ PIQMIVLTYF P 491 |

### EXAMPLES

The peptides, compositions and related methods and systems herein disclosed are further illustrated in the following examples, which are provided by way of illustration and are not intended to be limiting.

### Example 1

### In vitro Study About the Effect of the Dissociation of MDM4 from MDM2 on Cellular Death; Preparation of Peptides Suitable to Dissociate the Heterodimer and in vitro Study of Apoptotic Effectiveness Thereof

1.1 Materials and Methods

Tranfections. Transient transfections have been carried out using lipophilic vectors (“Lipofectamine 2000” for DNA, Invitrogen). This method is based on use of lipid vesicles (liposomes) mediating the transfer of DNA into eucaryotic cells.

Cells in 100 mm plates have been transfected with following vectors: pShutlle-hMDM4 (WT, C437G), pCMV-hMDM2 (WT), pCAG-p53.

Punctiform mutants have been obtained by PCR amplification with specific primers using plasmids containing hMdm4 and hMDM2 cDNAs and resulting products used for generation of plasmids containing hMdm4 and hMDM2 cDNAs using Qiagen mutagenesis kit.

Immunoprecipitation.

Cells have been lysed using Saito lysis modified reagent (50 mM Tris-HCl, pH 7.4, 0.15 M NaCl, 0.5% Triton-X100, 5 mM EDTA). For p53 immunoprecipitation and BCL2 and MDM4 analysis EBC modified solution has been used (50 mM Tris-HCl, pH 7.4, 0.12 M NaCl, 0.4% NP-40, 1 mM EDTA, 1 Mm β-mercaptoethanol).

400 μg of total protein extract has been incubated with protein G (Invitrogen) for 2 hours under stirring at 4° C. in order not specific binding of proteins with said substrate can occur. Afterwards the cellular extract has been centrifuged, not specific complexes removed with precipitate and supernatant has been incubated for 16 hours with specific antibody. Successively, protein G has been added and incubated for 2 hour at 4° C. Finally, immunoprecipitate has been washed twice with Saito buffer and resuspended in 10 μl of loading buffer solution (loading buffer 4×: 0.6 M β-mercaptoethanol; 8% SDS; 0.25 M Tris-HCl, pH 6.8; 40% glycerol; 0.2% Bromophenol blue). Samples have been denatured at 95° C. for 5 minutes before loading on gel for electrophoresis and successive western blot analysis.

Blot Western Analysis.

Different amounts (70-100 μg for samples to be analyzed) of total protein lysates have been separated by polyacrylamide gel electrophoresis (29% acrylamide and 1% bis-acrylamide) at 12% final concentration. Loading solution has been added to samples (loading buffer 4×: 0.6 M β-mercaptoethanol; 8% SDS; 0.25 M Tris-HCl, pH 6.8; 40% glycerol; 0.2% Bromophenol blue) suitable to denature definitively the same (as result of disruption of disulfide bridges by β-mercaptoethanol) and confer a definitive negative charge to all the proteins (by conjugation with SDS). Samples are boiled at 95° C. for 5 minutes and successively loaded on gel. Electrophoretic run has been carried out at 100 V for approximately 2 hours in ionic buffer containing 25 mM Tris-HCl, pH 8.8, 200 mM glycine and 0.1% SDS.

At the end of electrophoretic run proteins have been transferred on PVDF filter (Millipore) under electric field in buffer consisting of 25 mM Tris Base, 200 mM glycine and 20% methanol. Transfer of proteins from anode to cathode has been carried out for 3 hours at 70 V.

After proteins transfer, the filter has been incubated under stirring for 1 hour in TPBS buffer 1× (10 mM Tris-HCl, pH 7.6, 150 mM NaCl and 0.1% Tween 20) containing 5% skimmed milk powder (Bio-Rad) in order to block not specific antibody binding sites. The filter successively has been incubated for 2 hours with opportunely diluted primary antibody. At the end of the incubation the filter has been washed twice for approximately 10 minutes each with TPBS 1× and then incubated for 45 minutes with corresponding peroxidase-conjugated secondary antibody. After two washings for approximately 10 minutes with TPBS 1×, immunoreactivity has been evaluated using chemiluminescence reaction based on luminol oxidation (Amersham). Successively, chemiluminescence has been detected by autoradiography on sensitive plates (Kodak).

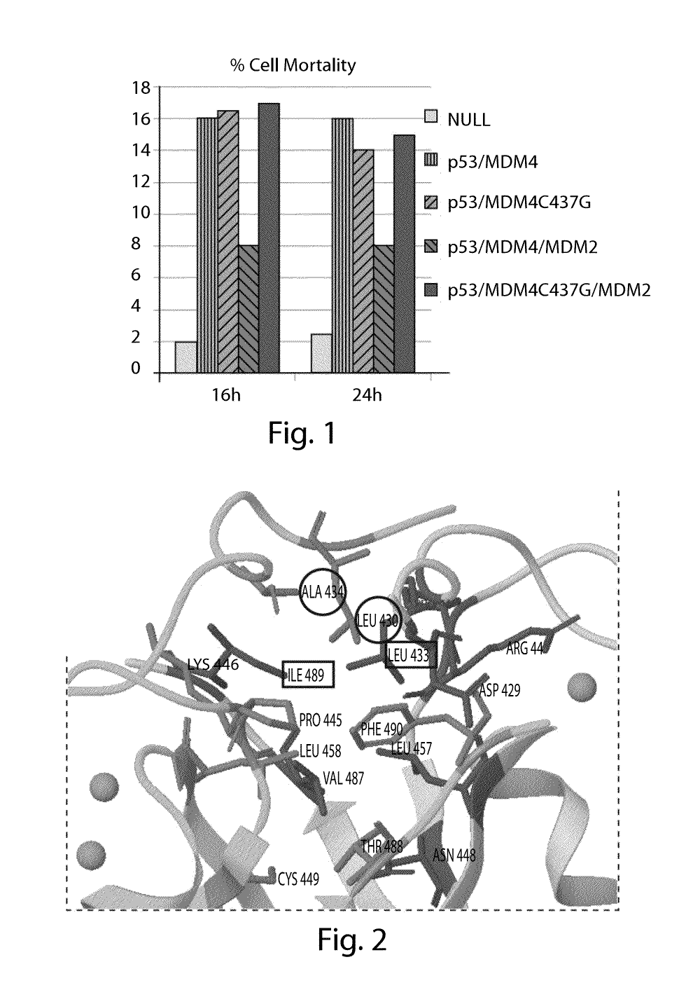

1.2 Effects of dissociation of MDM4 from MDM2 MDM2/MDM4 heterodimer is the main control tool for p53 levels and therefore inhibition thereof inside of cell under normal growth conditions (Wade and Wahl, 2009). Therefore it has been assumed that the dissociation of MDM4 from MDM2 can favor the implementation of pro-apoptotic function thereof, in addition to preventing degradation activity with respect to p53. The binding leading to heterodimer formation occurs at the level of “RING finger” domains of both the proteins (Tanimura et al., 1999). In order to point out the role of dissociation in the accomplishment of MDM4 pro-apoptotic activity, MDM4 mutant mutated at 437 cysteine residue has been used. Such mutation results in the impossibility to form a correct RING finger domain and thus MDM2 binding impossibility. Therefore, DNX cells have been transfected with constructs for expression of p53, MDM4 or MDM4C437G, and MDM2 proteins. Then Trypan Blue staining positive death cells have been counted and compared to number of total counted cells (FIG. 1). As it is apparent from obtained data MDM4 presence potentiates pro-apoptotic activity of p53, as previously observed and MDM4C437G expression acts analogously to MDM4-wt. However, MDM2 expression rescues cell death induced by co-expression of p53 and MDM4-wt, but not induced by MDM4C437G mutant. These data suggest, therefore, that factors suitable to prevent the association between MDM2 and MDM4 can avoid the functional inhibition of MDM2 and therefore potentiate the pro-apoptotic function of p53.

1.3 Preparation of Peptides Suitable to Disrupt the Interaction Between MDM2 and MDM4

For design of peptide suitable to disrupt the interaction between MDM2 and MDM4, initially an analysis at crystallized structure interfaces of MDM2 and MDM4 RING domains (pdb code: 2vjf) has been carried out. This analysis, in particular, has been carried out using a software program (SiteMap, Schrodinger Software) suitable to characterize binding pockets in proteins. As result, two small binding pockets at level of MDM2 and MDM4 N-terminal regions, respectively, have been detected.

Human MDM4 and MDM2 protein sequences, MDM4 and MDM2 amino acid sequences of MDM4 and MDM2 interaction domains and MDM4 and MDM2 amino acid sequences occurring in crystallographic structure of MDM4/MDM2 dimer are below reported:

| MDM4 human protein sequence | |
|---|---|
| (SEQ ID NO: 4) | |
| 1 | MTSFSTSAQC STSDSACRIS PGQINQVRPK LPLLKILHAA GAQGEMFTVK EVMHYLGQYI |
| 61 | MVKQLYDQQE QHMVYCGGDL LGELLGRQSF SVKDPSPLYD MLRKNLVTLA TATTDAAQTL |
| 121 | ALAQDHSMDI PSQDQLKQSA EESSTSRKRT TEDDIPTLPT SEHKCIHSRE DEDLIENLAQ |
| 181 | DETSRLDLGF EEWDVAGLPW WFLGNLRSNY TPRSNGSTDL QTNQDVGTAI VSDTTDDLWF |
| 241 | LNESVSEQLG VGIKVEAADT EQTSEEVGKV SDKKVIEVGK NDDLEDSKSL SDDTDVEVTS |
| 301 | EDEWQCTECK KFNSPSKRYC FRCWALRKDW YSDCSKLTHS LSTSDITAIP EKENEGNDVP |
| 361 | DCRRTISAPV VRPKDAYIKK ENSKLFDPCN SVEFLDLAHS SESQETISSM GEQLDNLSEQ |
| 421 | RTDTENMEDC QNLLKPCSLC EKRPRDGNII HGRTGHLVTC FHCARRLKKA GASCPICKKE |
| 481 | IQLVIKVFIA 490 |
| MDM4 sequence of MDM4-MDM2 interaction domain | |
| (SEQ ID NO: 5) | |
| 413 | QLDNLSEQ RTDTENMEDC QNLLKPCSLC EKRPRDGNII HGRTGHLVTC FHCARRLKKA |
| | GASCPICKKE IQLVIKVFIA 490 |
| MDM4 sequence occurring in crystallographic | |
| structure of MDM4/MDM2 dimer | |
| (SEQ ID NO: 6) | |
| 428 | EDC QNLLKPCSLC EKRPRDGNII HGRTGHLVTC FHCARRLKKA GASCPICKKE |
| | IQLVIKVFIA 490 |
| MDM2 human protein sequence | |
| (SEQ ID NO: 7) | |
| 1 | MCNTNMSVPT DGAVTTSQIP ASEQETLVRP KPLLLKLLKS VGAQKDTYTM KEVLFYLGQY |
| 61 | IMTKRLYDEK QQHIVYCSND LLGDLFGVPS FSVKEHRKIY TMIYRNLVVV NQQESSDSGT |
| 121 | SVSENRCHLE GGSDQKDLVQ ELQEEKPSSS HLVSRPSTSS RRRAISETEE NSDELSGERQ |
| 181 | RKRHKSDSIS LSFDESLALC VIREICCERS SSSESTGTPS NPDLDAGVSE HSGDWLDQDS |
| 241 | VSDQFSVEFE VESLDSEDYS LSEEGQELSD EDDEVYQVTV YQAGESDTDS FEEDPEISLA |
| 301 | DYWKCTSCNE MNPPLPSHCN RCWALRENWL PEDKGKDKGE ISEKAKLENS TQAEEGFDVP |
| 361 | DCKKTIVNDS RESCVEENDD KITQASQSQE SEDYSQPSTS SSIIYSSQED VKEFEREETQ |
| 421 | DKEESVESSL PLNAIEPCVI CQGRPKNGCI VHGKTGHLMA CFTCAKKLKK RNKPCPVCRQ |
| 481 | PIQMIVLTYF P |
| MDM2 sequence of MDM4-MDM2 interaction domain | |
| (SEQ ID NO: 8) | |
| 411 | VKEFEREETQ DKEESVESSL PLNAIEPCVI CQGRPKNGCI VHGKTGHLMA CFTCAKKLKK |
| | RNKPCPVCRQ PIQMIVLTYF P 491 |
| MDM2 sequence occurring in crystallographic | |
| structure of MDM4/MDM2 dimer | |
| (SEQ ID NO: 9) | |
| 428 | SSL PLNAIEPCVI CQGRPKNGCI VHGKTGHLMA CFTCAKKLKK RNKPCPVCRQ |
| | PIQMIVLTYF P 491 |

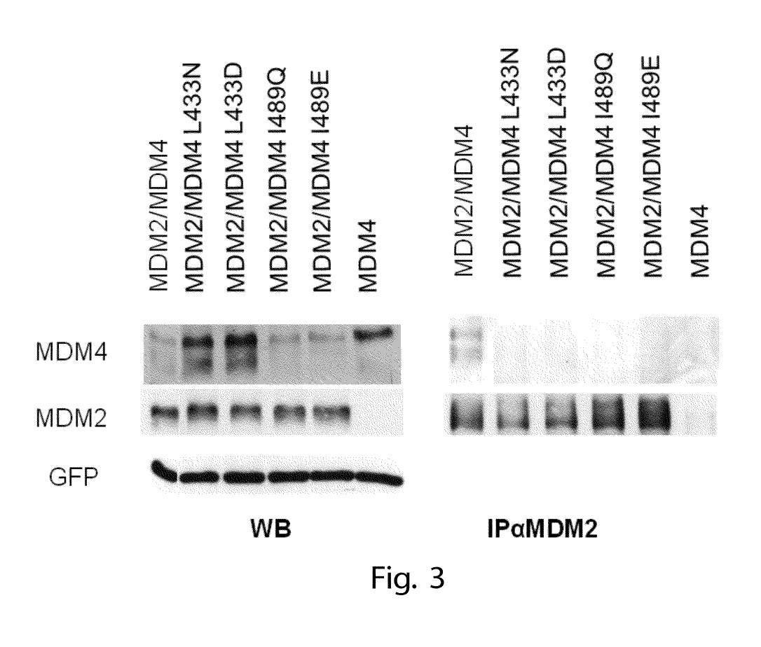

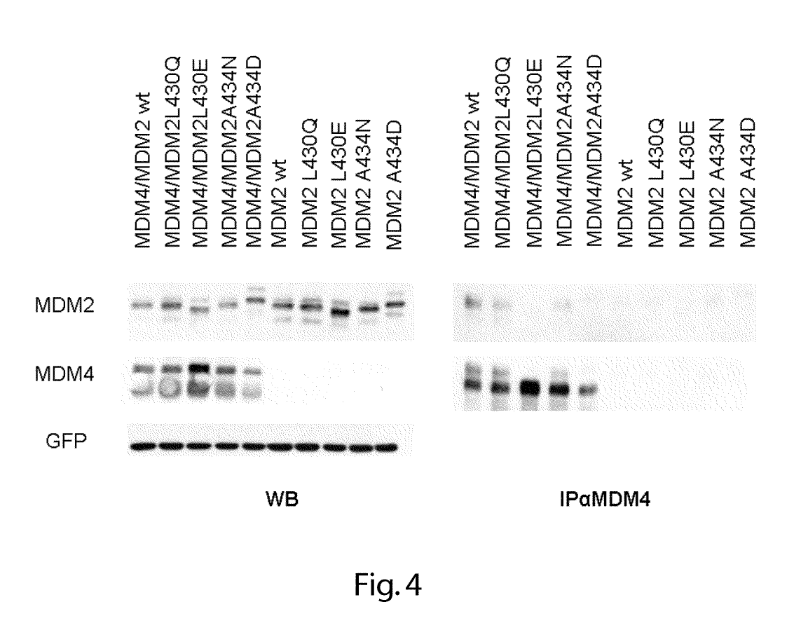

Starting from N-terminal regions of MDM2 and MDM4, the detection of key amino acid set involved in mediating interactions occurring at interface of MDM2/MDM4 complex has been carried out. Among these residues, Leu₄₃₀^{MDM2}, Ala₄₃₄^{MDM2}, Leu₄₃₃^{MDM4} and Ile₄₈₉^{MDM4} have been detected as particularly important for stabilization of heterodimeric complex of RING domain (FIG. 2). In order to prove that said amino acids carry out a determining role in interaction of two proteins, the same have been replaced with other amino acid residues by point mutagenesis experiments. Mutations have been carried out in such a way to replace every amino acid of interest with an amino acid unable to form the association but, at the same time, maintaining unaltered the correct “folding” of the corresponding domain and thus of the protein. Moreover, in order to prove that the cause of possible dissociation was effectively the replaced and not inserted amino acid, every amino acid residue has been replaced with two different residues with different total charge. Thus amino acids with net negative side chain charge, i.e. glutamate and aspartate, and corresponding polar side chain amino acids, i.e. glutamine and asparagine, have been selected. Accordingly, eight different mutants by in vitro mutagenesis have been produced:
1. MDM4L433N
2. MDM4L433D
3. MDM4I489Q
4. MDM4I489E
5. MDM2L430Q
6. MDM2L430E
7. MDM2A434N
8. MDM2A434D
   At this point, the ability of each mutant to interact with corresponding “wild-type” protein has been analyzed. DNX cells have been transfected with constructs expressing MDM2-wt protein and each of the four MDM4 mutants or MDM4-wt protein like control. Cells successively have been collected and resulting lysates immunoprecipitated with anti-MDM2 antibody and the immunocomplexes analyzed for the presence of MDM4 different forms (FIG. 3). Results have demonstrated that all and the four MDM4 mutants proved to be unable to bind MDM2, meanwhile it is very clear the binding of two “wild-type” proteins. This results indicates therefore that for MDM4 leucine in position 433 and isoleucine in position 489 are determinant for binding with MDM2 and therefore formation of heterodimer. In specular way, MDM2 mutants have been tested. Murine fibroplast cells derived from p53 and Mdm2 (DN1) “knockout” mouse have been transfected with constructs expressing MDM4 protein and each of different mutants of MDM2 or MDM2-wt like control. After 24 hours, cells have been collected and corresponding lysates immunoprecipitated using anti-MDM4 antibody (FIG. 4). According to western blot analysis of this immunoprecipitation it can be observed, as expected, that MDM2-wt binds MDM4 meanwhile MDM2 L430E and A434D mutants are unable to bind MDM4 under the same conditions. MDM2 L430Q and A434N mutants instead prove to be able to bind MDM4 although in lower extent than MDM2-wt protein. This result indicates therefore that 430 leucine and 434 alanine are two determining sites for MDM2 and MDM4 binding. However the insertion in these sites of an amino acid with weakly polar side chain clearly does not compromise remarkably the MDM2 ability to bind MDM4 while the insertion of amino acids with net negative side chain charge is suitable to dissociate the complex completely, suggesting that at level of these sites the bind is localized on side chain of involved amino acids. Starting from these residues, three MDM2 and MDM4 12mer peptides, corresponding to following amino acid sequences (SEQ ID NO: 1, SEQ ID NO: 2 and SEQ ID NO: 3), have been designed:

(SEQ ID NO: 1)
1) ESSLPLNAIEPC
i.e. Glu₁-Ser₂-Ser₃-Leu₄-Pro₅-Leu₆-Asn₇-
Ala₈-Ile₉-Glu₁₀-Pro₁₁-Cys₁₂
(SEQ ID NO: 2)
2) MEDCQNLLKPCS
i.e. Met₁-Glu₂-Asp₃-Cys₄-Gln₅-Asn₆-Leu₇-
Leu₈-Lys₉-Pro₁₀-Cys₁₁-Ser₁₂
(SEQ ID NO: 3)
3) KEIQLVIKVFIA
i.e. Lys₁-Glu₂-Ile₃-Gln₄-Leu₅-Val₆-Ile₇-
Lys₈-Val₉-Phe₁₀-Ile₁₁-Ala₁₂

Since the alpha helix conformation stability (helicity) is important parameter in order to favor a strong interaction between peptides and MDM2 and MDM4 RING domains, helicity of peptides 1 and 2 using simulations of molecular dynamics has been evaluated. These simulations have been carried out using Desmond software program and OPLS 2005 force field, as implemented according to Schrodinger software package. Particularly, simulations have carried out for 20 ns time length in aqueous solvent, at a temperature of 300° K, and according to NPT Berendesen protocol. Trajectories obtained by two simulations then have been analyzed through the calculation of secondary structure of peptides during said 20 ns. Resulting peptide helicities, expressed as percentage, has been 23.7% and 12.5% for peptide 1 and peptide 2, respectively. It has not been possible a similar analysis to be carried out for peptide 3.

1.4 Study on Apoptotic Effectiveness of Peptide 1 and Peptide 3

SEQ ID NO: 1-3 peptides have been modified by addition of FITC at N-terminal residue followed by a spacer. Amide group, in order to increase peptide stability, has been attached at C-terminal residue:

| | |
|---|---|
| Peptide 1: | FITC-Ahx-ESSLPLNAIEPS-CONH2 |
| Peptide 2: | FITC-Ahx-MEDSQNLLKPSS- CONH2 |
| Peptide 3: | FITC-Ahx-KEIQLVIKVFIA-CONH2 |

Ahx term indicates a linker.

Below properties thereof are shown.

Peptide 1 (SEQ ID NO: 1): the peptide has been tested by immunoprecipitation assays and detection of cell death.

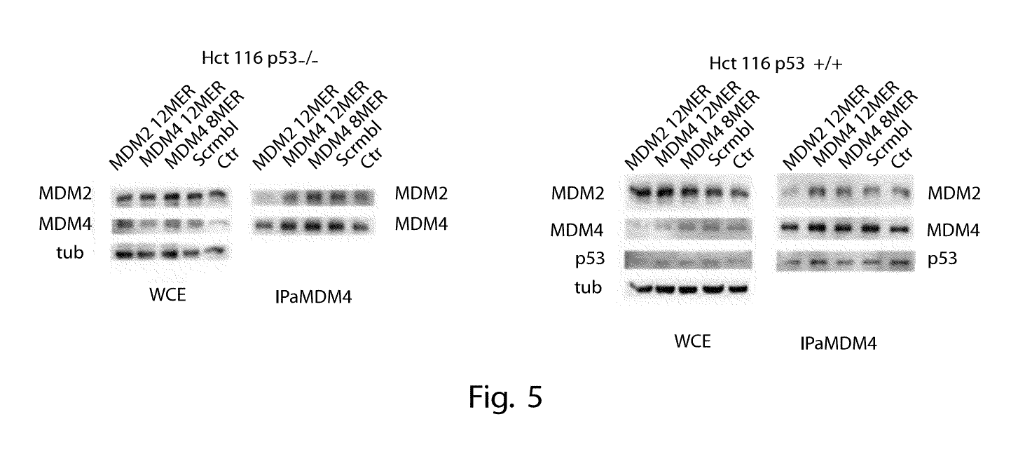

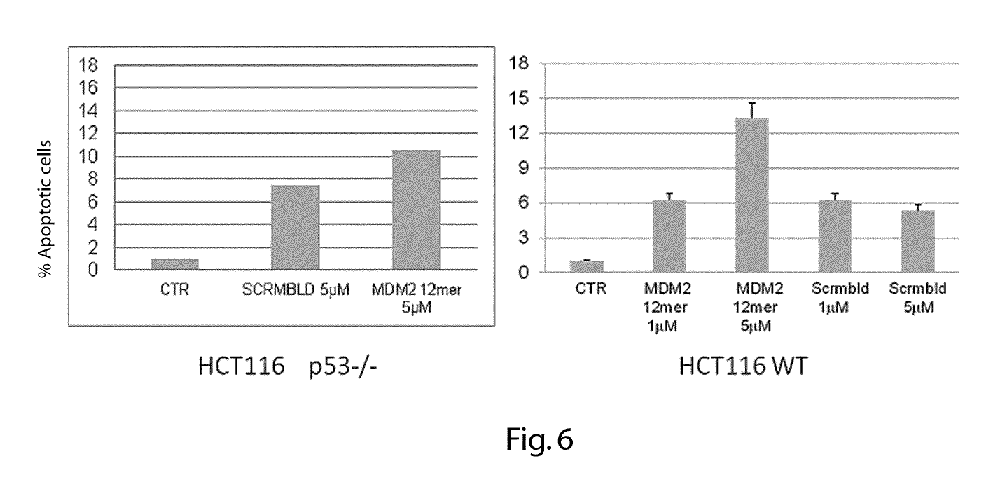

It has been investigated whether peptides were able to enter the cell simply by addition thereof at given concentration (5 μM) to cell culture medium and analyzing for the presence thereof by confocal and fluorescence microscopy, each peptide being labeled with green fluorophore (FAM). It has been observed that peptides were detected in cells only after transfection by liposomes. Successively, the ability of peptides to disrupt the binding by MDM4 or MDM2 immunoprecipitation after peptide transfection in HCT116 cancer cell line, and detection of presence of corresponding partner in immunocomplex by western blot assay have been evaluated. Two HCT116 wild type and HCT116 p53−/− syngeneic cell lines have been used in order to understand also observed effect dependence on the presence of p53. SEQ ID NO:1 peptide proved to be suitable to dissociate said two proteins and induce a significant apoptotic response (FIG. 5, FIG. 6). Of interest, this response was significantly higher in comparison to the control Scramble peptide in HCT116 p53+/+ cells but not in HCT116 p53−/− cells, indicating the specific activity of this peptide in the presence of p53 (FIG. 6). Accordingly, this peptide was able to preserve the binding of p53 to MDM4 (FIG. 5).

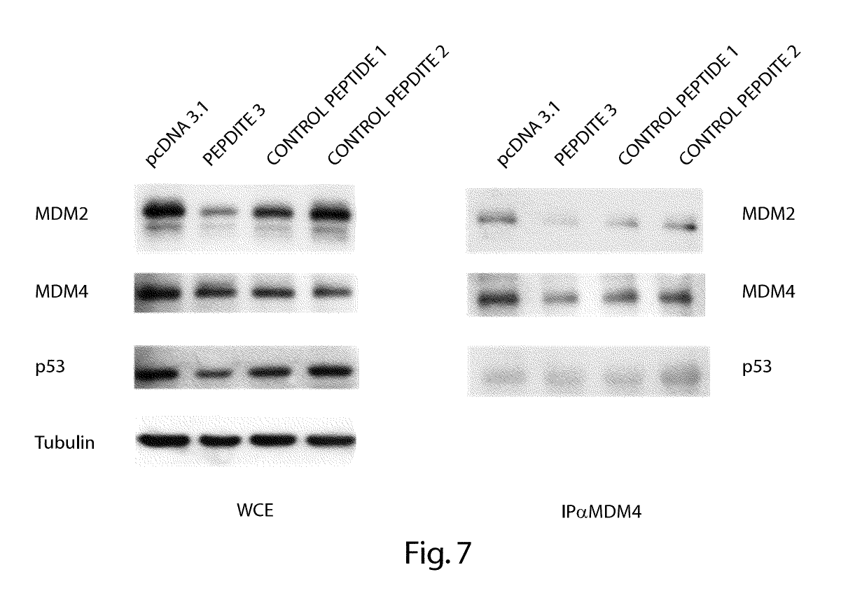

Peptide 3 (SEQ ID NO: 3): analogously to peptide 1, also for this peptide the ability to penetrate the cell has been evaluated by counting the number of cells positive for the presence of FITC fluorophore. This peptide has been proved to be internalized in cells both by liposomal vectors and independently although with lower efficiency. Successively, ability thereof to dissociate MDM4/MDM2 complex has been analyzed by immunoprecipitation experiments. Applicants have observed that the peptide is highly effective in dissociating said two molecules while does not dissociate p53 from MDM4 (FIG. 7).

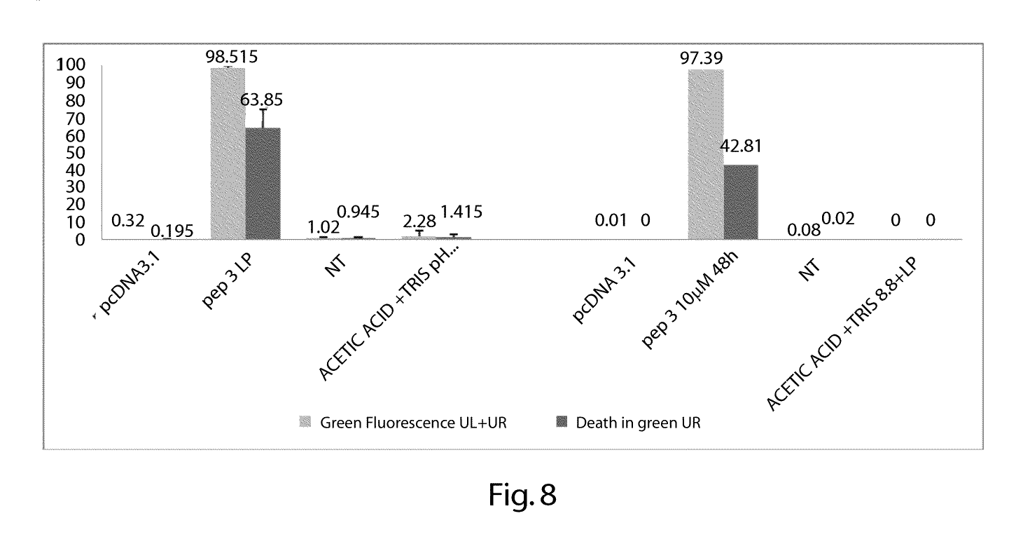

Moreover, this peptide has been proved to be highly effective in inducing cellular apoptosis in specific way since another peptide unable to dissociate MDM4/MDM2 does not induce cell death (not shown data). Apoptotic effect in addition is dependent on p53 since induced death is meaningfully lower in p53 lacking than harboring cells (FIG. 8).

### Example 2

### Study About the Role of Peptide 3 (SEQ ID NO:3) Composition in Its Molecular and Biological Activity

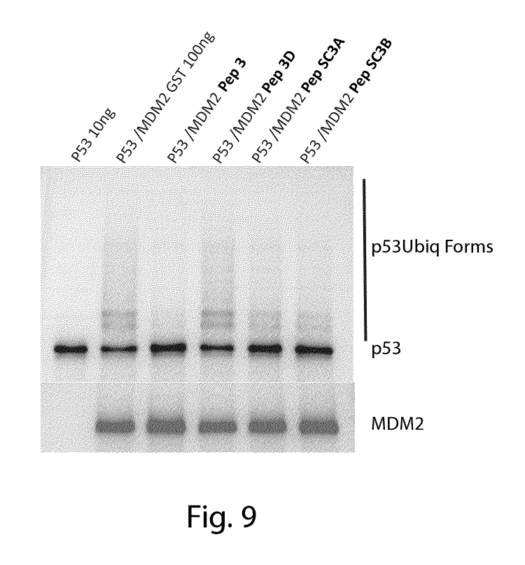

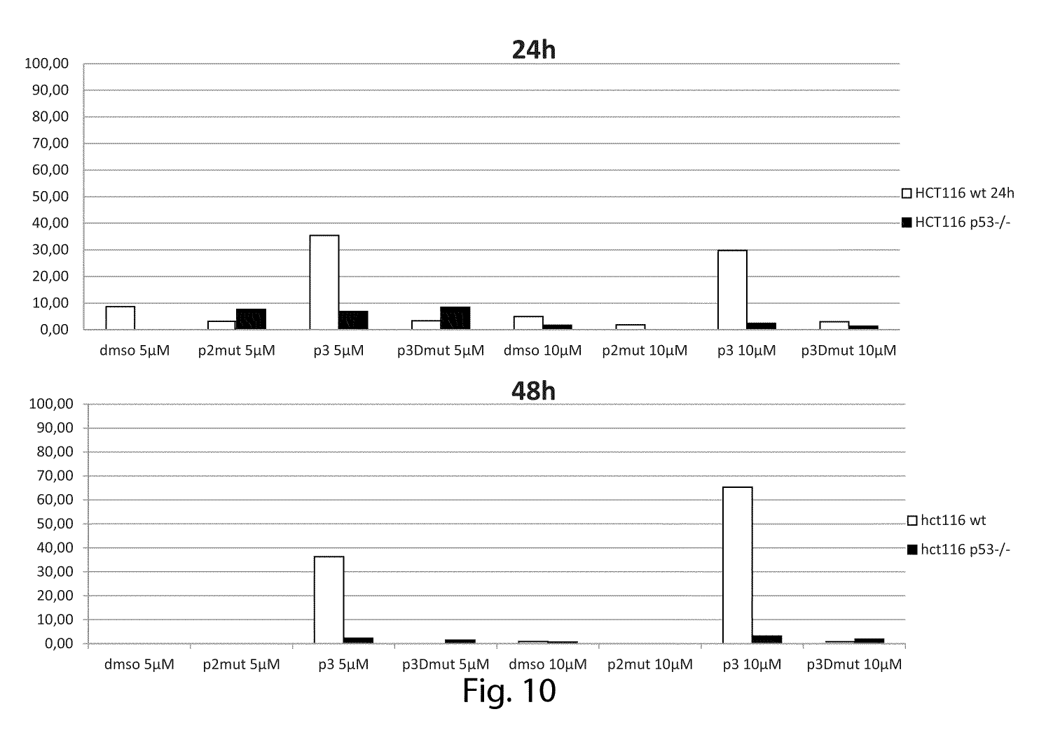

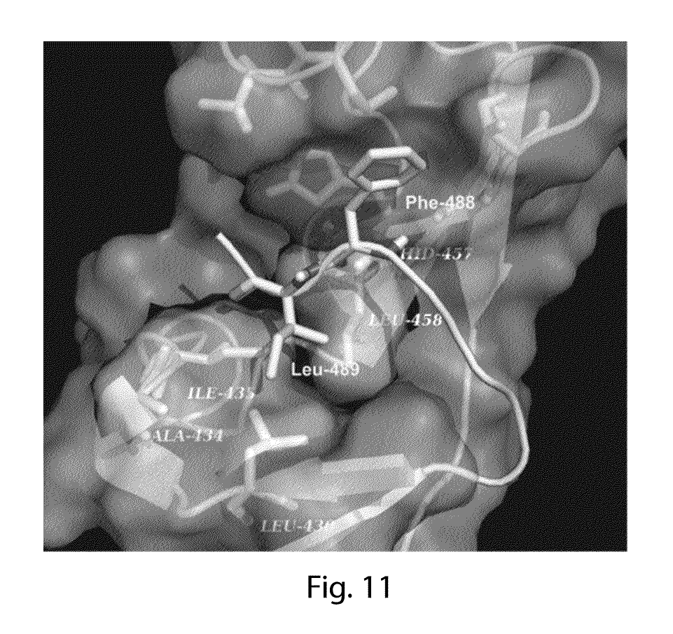

Further experiments have been performed to define the role of peptide 3 composition in its molecular and biological activity. These experiments have demonstrated that aminoacids Isoleucine at position 489 and Phenylalanine at position 488 of MDM4 protein are essential for the ability of Peptide 3 to inhibit MDM4/MDM2 activity and apoptosis-promoting function. Indeed, a peptide with two substitutions at position 10 and 11 of peptide 3 (Peptide 3D, KEIQLVIKVAEA (SEQ ID NO:38) was completely ineffective in inhibiting ubiquitination of p53 and inducing apoptosis (FIGS. 9 and 10). Similarly, two peptides composed with the same aminoacids of peptide 3 but arranged in different order (Peptide SC3A, VQEAFKLIKIVI (SEQ ID NO:39) and Peptide SC3B, AIKIFVKVLEIQ (SEQ ID NO:40) are ineffective in inhibiting ubiquitination of p53 (FIG. 9), confirming that the aminoacid Isoleucine at position 11 and Phenylalanine at position 10 are crucial contact points for inhibition of MDM4/MDM2 heterodimer activity. Supporting these new experimental data about the importance of Isoleucine 489 and Phenylalanine 488, molecular dynamic simulations have shown that these residues engage in specific hydrophobic and aromatic interactions with MDM2 (FIG. 11). In particular, Isoleucine 489 is harbored in a hydrophobic cleft composed of Alanine 434, Isoleucine 435 and Leucine 458 of MDM2 protein. Likewise, Phenylalanine 488 forms aromatic interactions with a Histidine residue (HID-457) of MDM2 protein.

In addition, since SC3A and SC3B have different solubility and helical content (as estimated by AGADIR software), these data reinforce the importance of targeting aa A488 and 1489, independently of other peptide properties.

The examples set forth above are provided to give those of ordinary skill in the art a complete disclosure and description of how to make and use the embodiments of the peptides, compositions, arrangements, devices, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, Examples and Annex A is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

Further the sequence listing filed concurrently with present description as txt file P1202-US-2013-02-21-Sequence Listing_ST25, forms integral part of the present disclosure and is incorporated herein by reference in its entirety.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations which is not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims

1. Peptide or a mimetic thereof, the peptide being able to interfere with an MDM2/MDM4 interaction domain comprising an MDM2 sequence and an MDM4 sequence, the peptide having from 5 to 16 amino acid residues with a sequence from the MDM2 sequence, or the MDM4 sequence,
wherein
the MDM2 sequence comprises amino acids from position 428 to position 491 of MDM2 protein and the MDM4 sequence comprises amino acids from position 413 to position 490 of MDM4 protein
wherein
the peptide from the MDM2 sequence comprises at least one of amino acids leucine or alanine of MDM2 position 430 and 434 respectively, and the MDM2 sequence comprises sequence EREETQ DKEESVESSL PLNAIEPCVI CQGRPKNGC (SEQ ID NO:12) from position 415 to position 449;
and wherein
the peptide from the MDM4 sequence comprises at least one of amino acids leucine, isoleucine or phenylalanine of MDM4 positions 433, 489 or 488, respectively,
the MDM4 sequence comprises sequence SCPICKKE IQLVIKVFIA (SEQ ID NO: 10) from position 473 to position 490; and/or
the MDM4 sequence comprises sequence RTDTENMEDC QNLLKPCSLC EKRPRDGN (SEQ ID NO: 11) from position 418 to position 448.

2. The peptide or mimetic thereof according to claim 1, wherein, the peptide has a sequence from the MDM4 and comprises both amino acids isoleucine and phenylalanine of MDM4 positions 489 and 488 respectively.

3. The peptide or mimetic according to claim 1, wherein the peptide has a sequence consisting of the MDM2 sequence from amino acid 427 to amino acid 438 or a sequence of a mimetic peptide thereof.

4. The peptide or mimetic according to claim 1, wherein the peptide has a sequence consisting of the MDM4 sequence from amino acid 479 to amino acid 490 or a sequence of a mimetic peptide thereof.

5. The peptide or mimetic according to claim 1, wherein the peptide has 8 to 12 amino acid residues.

6. Peptide or mimetic able to interfere with an MDM2/MDM4 interaction domain, wherein the peptide has the following general formula I:
R-KVFI-R1 (I)
in which
R is selected from the group consisting of I, VI, LVI, QLVI (SEQ ID NO:13), IQLVI (SEQ ID NO:14), EIQLVI (SEQ ID NO:15), KEIQLVI (SEQ ID NO:16), KKEIQLVI (SEQ ID NO:17), CKKEIQLVI (SEQ ID NO:18), ICKKEIQLVI (SEQ ID NO:19), PICKKEIQLVI (SEQ ID NO:20), CPICKKEIQLVI (SEQ ID NO:21) and hydrogen of amino functional group of K;
R1 is selected from the group consisting of A and hydroxyl of carboxylic functional group of I;
wherein said R and R1 are chosen so that the peptide ranges from 5 to 16 amino acid.

7. The peptide or mimetic according to claim 6, wherein the peptide has sequence KEIQLVIKVFIA (SEQ ID NO:3)

8. The peptide or mimetic according to claim 6, wherein the peptide has 8 to 12 amino acid residues.

9. A peptide or mimetic according able to interfere with an MDM2/MDM4 interaction domain, wherein the peptide has the following general formula II:
R2-LPLNA-R3 (II)
wherein
R2 is selected from the group consisting of S, SS, ESS, VESS (SEQ ID NO:22), SVESS (SEQ ID NO:23), ESVESS (SEQ ID NO:24), EESVESS (SEQ ID NO:25), KEESVESS (SEQ ID NO:26), DKEESVESS (SEQ ID NO:27), QDKEESVESS (SEQ ID NO:28), TQDKEESVESS (SEQ ID NO:29) and hydrogen of amino functional group of L;
R3 is selected from the group consisting of I, IE, IEP, IEPC (SEQ ID NO:30), IEPCV (SEQ ID NO:31), IEPCVI (SEQ ID NO:32), IEPCVIC (SEQ ID NO:33), IEPCVICQ (SEQ ID NO:34), IEPCVICQG (SEQ ID NO:35), IEPCVICQGR (SEQ ID NO:36), IEPCVICQGRP (SEQ ID NO:37) and hydroxyl of carboxylic functional group of A;
wherein said R2 and R3 are chose so that said peptide ranges from 5 to 16 amino acids.

10. The peptide or mimetic according to claim 9, wherein the peptide has sequence ESSLPLNAIEPC (SEQ ID NO:1)

11. The peptide or mimetic according to claim 9, wherein the peptide has 8 to 12 amino acid residues.

12. An isolated nucleotide sequence codifying the peptide according to claim 1.

13. An expression vector comprising the nucleotide sequence according to claim 9.

14. A pharmaceutical composition comprising at least one of the peptide or mimetic thereof according to claim 1, at least one nucleotide sequence codifying for said at least one peptide or mimetic thereof, and/or at least one vector comprising said nucleotide sequence, the at least one peptide, at least one nucleotide sequence and/or at least one vector comprised as an active ingredient, in association with one or more pharmaceutically acceptable adjuvant and/or excipients.

15. A method to treat a tumor in an individual, the method comprising
administering to the individual in an effective amount to treat the tumor, at least one of the peptide or mimetic thereof according to claim 1, at least one nucleotide sequence codifying for said at least one peptide or mimetic thereof, and/or at least one vector comprising said nucleotide sequence.

16. The method of claim 15, wherein the tumor is a tumor associated with expression of wild type p53.

17. A method to treat a hyperproliferative benign syndrome in an individual, the method comprising
administering to the individual in an effective amount to treat the hyperproliferative benign syndrome, at least one of the peptide or mimetic thereof according to claim 1, at least one nucleotide sequence codifying for said at least one peptide or mimetic thereof, and/or at least one vector comprising said nucleotide sequence.

18. A method to screen a compound able to dissociate an MDM2/MDM4 interaction domain comprising an MDM2 sequence and an MDM4 sequence, the method comprising
contacting a candidate peptide (peptide or small molecule) with an MD2/MDM4 interaction domain in which
in the MDM2 sequence at least one of amino acids in position 430 and 434 is leucine or alanine respectively, and
in the MDM4 sequence at least one of amino acids in positions 433, 489 or 488, is leucine, isoleucine or phenylalanine respectively; and
detecting association or dissociation of MDM2/MDM4 interaction domain.

19. The method of claim 18, wherein the MDM4 sequence is QLDNLSEQ RTDTENMEDC QNLLKPCSLC EKRPRDGNII HGRTGHLVTC FHCARRLKKA GASCPICKKE IQLVIKVFIA (SEQ ID NO:5)

20. The method of claim 18, wherein the MDM4 sequence consists of an MDM4 sequence from amino acid 428 to amino acid 490

21. The method of claim 20, wherein the MDM4 sequence is 428 EDC QNLLKPCSLC EKRPRDGNII HGRTGHLVTC FHCARRLKKA GASCPICKKE IQLVIKVFIA 490 (SEQ ID NO:6)

22. The method of claim 18, wherein the MDM2 sequence is 411 VKEFEREETQ DKEESVESSL PLNAIEPCVI CQGRPKNGCI VHGKTGHLMA CFTCAKKLKK RNKPCPVCRQ PIQMIVLTYF P 491 (SEQ ID NO:8)

23. The method of claim 18, wherein the MDM2 sequence is 428 SSL PLNAIEPCVI CQGRPKNGCI VHGKTGHLMA CFTCAKKLKK RNKPCPVCRQ PIQMIVLTYF P 491 (SEQ ID NO:9)
